(19) United States
(12) Patent Application Publication
(10) Pub. No.: US 2008/0118524 A1
(43) Pub. Date: May 22, 2008

(54) Anti-IgE Vaccines

(76) Inventors: Stefan Persson, Uppsala (SE); Vidar Wendel-Hansen, Uppsala (SE)

Correspondence Address: FISH & RICHARDSON P.C., PO BOX 1022, MINNEAPOLIS, MN, 55440-1022, US

(21) Appl. No.: 11/551,510

(22) Filed: Oct. 20, 2006

(51) Int. Cl. A61K 39/00; C07K 16/42

(52) U.S. Cl. 424/185.1; 530/387.3

(57) ABSTRACT

Materials and methods for inducing an IgG anti-IgE response in a human. The methods can include administering to a human subject a polypeptide comprising a human CH3 domain of IgE located between an opossum CH2 domain of IgE and an opossum CH4 domain of IgE, wherein the polypeptide is administered at a dose from about 30 μg to about 600 μg.

### BACKGROUND

1. Technical Field

This document relates to methods and materials related to the use of a chimeric IgE polypeptide to elicit an anti-human IgE response in humans.

2. Background Information

During the past few decades, several diseases caused by malfunctions of the immune system have become the major challenges of modern day medicine. Two such areas are the allergic and autoimmune diseases. Allergies have become almost epidemic during the past 20-30 years. Estimates range from 20-30 percent of the total population being affected. Atopic allergies, or IgE mediated allergies, are the dominating form.

Common types of atopic allergies include hay fever, fur allergies, dust mite allergies, insect venom allergies, extrinsic asthma, and many types of food allergies. An interesting question is whether vaccines can be developed against these types of diseases. Hyposensitization therapy has been used to treat allergies since the beginning of the twentieth century (Noon, Lancet, 1:1572 (1911); and Freeman, Lancet, 1:1178 (1914)). This is an allergen-dependent treatment strategy, which involves the use of allergen extracts to treat patients by injection. Hyposensitization therapy has, however, been questioned due to often low efficacy and sometimes severe side effects. In addition, different extracts must be used for each individual form of allergy. New strategies to treat allergies thus are presently being evaluated.

### SUMMARY

This document provides materials and methods related to vaccines against human polypeptides. For example, this document provides compositions containing a chimeric IgE polypeptide and, optionally, an adjuvant. The polypeptide contains human components as well as components from non-placental mammals, which can result in anti-human immune responses when administered to a human subject. For example, when administered to a human, the chimeric IgE polypeptides provided herein can reduce the IgE antibody effects of IgE-related diseases such as asthma, allergies, and eczema. When included, the adjuvant typically is selected to give a relatively high antibody response to human IgE, as compared to compositions containing other adjuvants.

In one aspect, this document features a method for inducing an anti-human IgE antibody response in a human subject, the method including administering to the subject a polypeptide under conditions wherein the subject produces an anti-human IgE antibody response, wherein the polypeptide contains an amino acid sequence from a human IgE polypeptide and an amino acid sequence from an IgE polypeptide found in a non-placental mammal (e.g., an opossum), and wherein the polypeptide is administered in an amount from about 10 μg to about 600 μg (e.g., from about 30 μg to about 600 μg, from about 100 μg to about 500 μg, from about 200 μg to about 400 μg, from about 250 μg to about 350 μg, from about 400 μg to about 600 μg, from about 450 μg to about 550 μg, about 300 μg, or about 500 μg). The polypeptide can include an amino acid sequence from a human IgE CH3 domain located between amino acid sequences from opossum IgE CH2 and CH4 domains. The polypeptide can have the amino acid sequence set forth in SEQ ID NO:2.

The method can be used to induce a reversible anti-human IgE response in the human. For example, a polypeptide having a human IgE sequence can be administered to a human subject under conditions wherein the subject mounts an antibody response to human IgE in a manner such that the response peaks and then decreases with time. The anti-human IgE response can be a primary response that decreases with time (e.g., decreases to undetectable levels within 1, 2, 3, 4, 5, 6, 7, 8, 9, 10, 11, 12, or more months). The method can be used to induce an anti-human IgE response in a human subject after the subject has experienced a primary anti-human IgE response. For example, a polypeptide having a human IgE sequence can be administered to the subject under conditions wherein the subject mounts an antibody response to human IgE in a manner consistent with a secondary antibody response. The method can be used to induce a series of anti-human IgE responses in a human subject. For example, a method can include administering a polypeptide having a human IgE sequence to the subject at different times and/or under different conditions, wherein the subject mounts a detectable anti-human IgE response that peaks within at least one year (e.g., within at least 11, 10, 9, 8, 7, 6, 5, 4, 3, 2, or 1 month) of each administration.

This document also features a composition containing from about 10 μg to about 600 μg (e.g., from about 30 μg to about 600 μg, from about 100 μg to about 500 μg, from about 200 μg to about 400 μg, from about 250 μg to about 350 μg, from about 400 μg to about 600 μg, from about 450 μg to about 550 μg, about 300 μg, or about 500 μg) of a chimeric IgE polypeptide. The polypeptide can include an amino acid sequence from a human IgE CH3 domain located between amino acid sequences from opossum IgE CH2 and CH4 domains. The polypeptide can have the amino acid sequence set forth in SEQ ID NO:2.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph plotting IgG anti-OSO titers measured by ELISA. Median±interquartile ranges are plotted for each dose group. Only individuals administered active substance are included (n=6, except for dose group 4 week 10 where n=3), individuals that received placebo had no detectable IgG anti-OSO titers.

FIG. 2a is a graph plotting individual IgG anti-OSO titers measured by ELISA. Dose group 1 (10 μg). (P)=placebo. Subject 102 dropped out of the study and was not replaced. n=7

FIG. 2b is a graph plotting individual IgG anti-OSO titers measured by ELISA. Dose group 2 (30 μg). (P)=placebo.

FIG. 2c is a graph plotting individual IgG anti-OSO titers measured by ELISA. Dose group 3 (100 μg). (P)=placebo.

FIG. 2d is a graph plotting Individual IgG anti-OSO titers measured by ELISA. Dose group 4 (300 μg). (P)=placebo.

FIG. 3a is a graph plotting individual OD values (mean of duplicates) at dilution 1:90. Dose group 1 (10 μg). (P)=placebo.

FIG. 3b is a graph plotting individual OD values (mean of duplicates) at dilution 1:90. Dose group 2 (30 μg). (P)=placebo.

FIG. 3c is a graph plotting individual OD values (mean of duplicates) at dilution 1:90. Dose group 3 (100 μg). (P)=placebo.

FIG. 3d is a graph plotting individual OD values (mean of duplicates) at dilution 1:90. Dose group 4 (300 μg). (P)=placebo.

FIG. 4a is a graph plotting Biacore data of dose group 1 (10 μg). The average response±SD of single individuals over time was analyzed in quadruplicates and is presented as quotas. Response/LOD>1 is considered positive. (P)=placebo.

FIG. 4b is a graph plotting Biacore data of dose group 2 (30 μg). The average response±SD of single individuals over time was analyzed in quadruplicates and is presented as quotas. Response/LOD>1 is considered positive. (P)=placebo.

FIG. 4c is a graph plotting Biacore data of dose group 3 (100 μg). The average response±SD of single individuals over time was analyzed in quadruplicates and is presented as quotas. Response/LOD>1 is considered positive. (P)=placebo.

FIG. 4d is a graph plotting Biacore data of dose group 4 (300 μg). The average response±SD of single individuals over time was analyzed in quadruplicates and is presented as quotas. Response/LOD>1 is considered positive. (P)=placebo.

FIG. 5a is a graph plotting total and free (unbound) IgE (U/mL) in dose group 1 (10 μg RES 08). (P)=placebo.

FIG. 5b is a graph plotting total and free (unbound) IgE (U/mL) in dose group 2 (30 μg RES 08). (P)=placebo.

FIG. 5c is a graph plotting total and free (unbound) IgE (U/mL) in dose group 3 (100 μg RES 08). (P)=placebo.

FIG. 5d is a graph plotting total and free (unbound) IgE (U/mL) in dose group 4 (300 μg RES 08). (P)=placebo.

FIG. 6a is a nucleic acid sequence listing of an insert sequence that encodes an OSO polypeptide (SEQ ID NO:1). The OSO polypeptide contains an opossum CH2 IgE domain followed by a human CH3 IgE domain followed by an opossum CH4 IgE domain.

FIG. 6b is an amino acid sequence listing of an OSO polypeptide (SEQ ID NO:2).

### DETAILED DESCRIPTION

This document provides methods and materials related to vaccines against human IgE polypeptides. For example, this document provides compositions containing a polypeptide that contains human IgE components and IgE components from a non-placental mammal. Administration of such a polypeptide to a human subject can result in both anti-human and anti non-placental mammal immune responses in the subject. In some cases, a composition can contain a polypeptide and an adjuvant selected to give a relatively high antibody response against human IgE, as compared to compositions containing other adjuvants.

The term “polypeptide” as used herein refers to a chain of amino acids, regardless of length or posttranslational modification (e.g., phosphorylation or glycosylation). For example, in some embodiments, the polypeptide can be unmodified such that it lacks modifications such as phosphorylation and glycosylation. The polypeptide can contain part or all of a single naturally-occurring polypeptide, or can be a chimeric polypeptide containing amino acid sequences from two or more naturally-occurring polypeptides. An “adjuvant” is an immunological compound that can enhance an immune response against a particular antigen such as a polypeptide. Typically, the compositions provided herein are administered to a human subject such that the subject produces antibodies against the polypeptide component of the administered composition.

The compositions provided herein can elicit an anti-human IgE antibody response in a human subject. For example, a polypeptide can contain a human IgE polypeptide segment and one or more (e.g., two or three) IgE polypeptide segments from a non-placental mammal (e.g., an opossum, platypus, koala, wallaby, kangaroo, or wombat). The term “human” as used herein with reference to a polypeptide sequence refers to an amino acid sequence that is identical or similar to a sequence from humans in general, or from a human subject to which the composition is to be administered. A polypeptide can be, for example, an OSO polypeptide that contains sequences from the human and opossum IgE molecules, and can be administered to a human as described herein.

The human segment or segments, as well as the non-placental mammal segment or segments, can have any length, and typically are at least 5 amino acids in length (e.g., at least about 5, 10, 20, 30, 40, 50, 60, 65, 70, 75, 80, 85, 90, 95, 100, 105, 110, 115, 120, 125, 150, 175, 200, 500, 750, 1000, 2000, 3000, 4000, 5000, or more amino acids in length). For example, the human segment or segments, as well as the non-placental mammal segment or segments, can have a length ranging from about 20, 30, 40, 50, 60, 70, or 80 amino acids to about 90, 100, 110, 120, 130, 140, 150, 200, 250, or 500 amino acids.

Any method can be used to obtain a polypeptide. For example, molecular cloning techniques can be used to prepare a nucleic acid construct encoding a polypeptide containing human IgE segments and IgE segments from a non-placental mammal (e.g., OSO). Such a construct can be expressed in an organism such as E. Coli or S. cerevisiae, or in a cell line, for example, and then can be purified from cellular extracts or from culture supernatants. Alternatively, a polypeptide can be chemically synthesized.

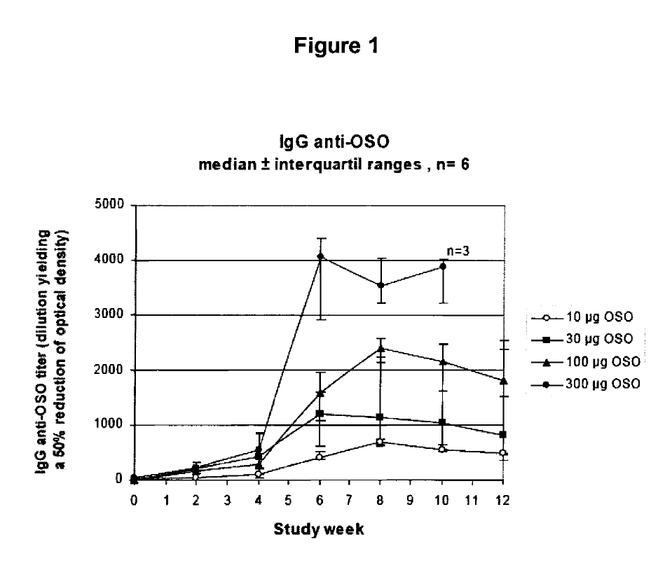

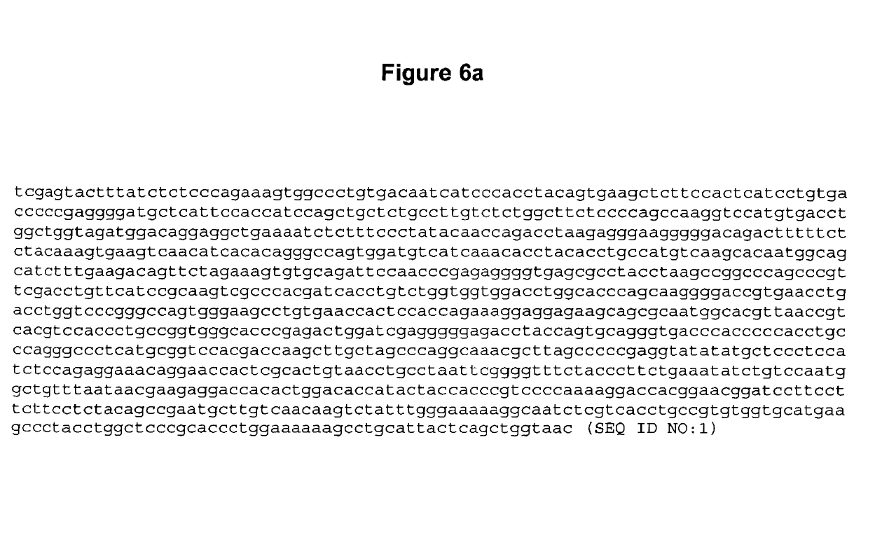

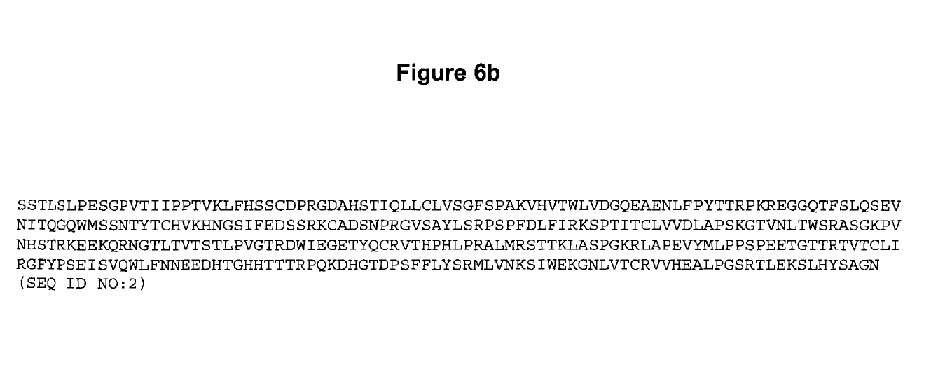

In particular, nucleic acid vectors can be designed to express chimeric IgE polypeptides. Examples of such nucleic acid vectors include, without limitation, those set forth in FIGS. 1, 2, 5, and 6 of U.S. Publication No. 20040054146. In addition, nucleic acid vectors can contain an insert sequence. The term “insert sequence” as used herein refers to a nucleic acid sequence that is inserted into a nucleic acid vector such that that inserted nucleic acid sequence can be expressed. An insert sequence can be a nucleic acid sequence that encodes a chimeric IgE polypeptide such as a polypeptide having the amino acid sequence set forth in FIG. 6b herein (SEQ ID NO:2). Such nucleic acid sequences can be as set forth in FIG. 6a herein (SEQ ID NO:1). The term “chimeric IgE polypeptide” as used herein refers to a polypeptide having a combination of IgE sequences (e.g., full domains, half domains, or quarter domains) from different species (e.g., humans and non-placental mammals). A chimeric IgE polypeptide typically contains IgE constant heavy (CH) chain domains (e.g., CH1, CH2, CH3, or CH4). For example, an insert sequence having the sequence set forth in SEQ ID NO:1 (FIG. 6a) can encode an opossum CH2-human CH3-opossum CH4 (ORO) chimeric IgE polypeptide (SEQ ID NO:2; FIG. 6b).

A nucleic acid vector also can contain components that affect expression of the insert sequence. Examples of such components include, without limitation, promoter, enhancer, leader, and polyadenylation sequences. Such components can be operably linked to the insert sequence. The term “operably linked” as used herein refers to an arrangement where components so described are configured so as to perform their usual function. For example, a nucleic acid vector with an insert sequence encoding an OSOSO chimeric IgE polypeptide also can contain a cytomegalovirus (CMV) promoter sequence (see, for example, Thomson et al., Proc. Natl. Acad. Sci. U.S.A., 81(3):659-663, 1984), an immunoglobulin (Ig) leader sequence (see, for example, Neuberger et al., EMBO J., 2(8):1373-1378, 1983), and a bovine growth hormone (bGH) polyadenylation sequence (see, for example, Goodwin et al., J. Biol. Chem., 267:16330-16334, 1992). In this case, the components can be operably linked to the insert sequence such that the CMV promoter can drive the expression of the insert sequence including the Ig leader sequence and bGH polyadenylation sequence, the Ig leader sequence can direct the expressed insert sequence into the lumen of the endoplasmic reticulum in preparation for secretion, and the bGH polyadenylation sequence can stabilize the insert sequence transcript.

In addition, a nucleic acid vector can contain components that aid in the growth, maintenance, or selection of a host cell containing the nucleic acid vector. Such components include, without limitation, origins of replication and antibiotic selection markers. For example, a nucleic acid vector with a CMV promoter sequence, an Ig leader sequence, an SV40 late polyadenylation sequence, and an insert sequence encoding an OSO chimeric IgE polypeptide can also contain an f1 origin of replication sequence, a sequence that confers ampicillin resistance on a bacterial host cell when expressed, and a sequence that confers neomycin resistance on a mammalian host cell when expressed. Other examples of antibiotic selection markers include, without limitation, sequences that confer resistance to hygromycin B, puromycin, kanamycin, tetracycline, blasticidin S, Geneticin®, and zeocin on a host cell when expressed. Nucleic acid vectors that contain one or more than one component described herein can be obtained commercially from, for example, Invitrogen (Carlsbad, Calif.) and Promega (Madison, Wis.).

Polypeptides containing human IgE sequences can be obtained using host cells containing a nucleic acid vector (e.g., the pCI-neo vector from Promega, catalogue number E1841) with an insert sequences provided herein (e.g., OSO). Such cells can be prokaryotic cells (e.g., JM109 or DH5α cells) or eukaryotic cells (e.g., NSO, HeLa, BHK-21, COS-7, Sf9, or CHO cells). Host cells containing the nucleic acid vector may or may not express the encoded polypeptide. For example, a host cell may function simply to propagate the nucleic acid vector for use in other host cells. In addition, the nucleic acid vector can be integrated into the genome of the host or maintained in an episomal state. Thus, a host cell can be stably or transiently transfected with the nucleic acid vector.

A host cell can contain a nucleic acid vector with an insert sequence that encodes a chimeric IgE polypeptide. For example, a host cell can contain a nucleic acid vector with an insert sequence encoding an OSO chimeric IgE polypeptide as provided herein. In addition, a host cell can express the polypeptide encoded by the insert sequence.

Various methods can be used to introduce a nucleic acid vector into a host cell in vivo or in vitro. For example, calcium phosphate precipitation, electroporation, heat shock, lipofection, microinjection, and viral-mediated nucleic acid transfer are common methods that can be used to introduce a nucleic acid vector into a host cell. In addition, naked DNA can be delivered directly to cells in vivo as described elsewhere (U.S. Pat. Nos. 5,580,859 and 5,589,466). Further, a nucleic acid vector can be introduced into cells to generate transgenic animals.

Transgenic animals can be aquatic animals (such as fish, sharks, dolphin, and the like), farm animals (such as pigs, goals, sheep, cows, horses, rabbits, and the like), rodents (such as rats, guinea pigs, and mice), non-human primates (such as baboon, monkeys, and chimpanzees), and domestic animals (such as dogs and cats). Several techniques known in the art can be used to introduce a nucleic acid vector into animals to produce the founder lines of transgenic animals. Such techniques include, without limitation, pronuclear microinjection (U.S. Pat. No. 4,873,191); retrovirus mediated gene transfer into germ lines (Van der Putten et al., Proc. Natl. Acad. Sci., USA, 82:6148 (1985)); gene transfection into embryonic stem cells (Gossler A et aL, Proc Natl Acad Sci USA 83:9065-9069 (1986)); gene targeting into embryonic stem cells (Thompson et al., Cell, 56:313 (1989)); nuclear transfer of somatic nuclei (Schnieke A E et al., Science 278:2130-2133 (1997)); and electroporation of embryos (Lo C W, Mol. Cell. Biol., 3:1803-1814 (1983)). Once obtained, transgenic animals can be replicated using traditional breeding or animal cloning.

Various methods can be used to identify a host cell containing a nucleic acid vector provided herein. Such methods include, without limitation, PCR, nucleic acid hybridization techniques such as Northern and Southern analysis, and in situ nucleic acid hybridization. In some cases, immunohistochemistry and biochemical techniques can be used to determine if a cell contains a nucleic acid vector with a particular insert sequence by detecting the expression of a polypeptide encoded by that particular insert sequence.

Any method can be used to produce recombinant chimeric IgE polypeptides. Such methods involve culturing a host cell that expresses a chimeric IgE polypeptide and recovering the expressed chimeric IgE polypeptides. Any method can be used to recover a recombinant chimeric IgE polypeptide. For example, recombinant chimeric IgE polypeptides that are present in a host cell homogenate can be recovered using ion exchange chromatography. In another example, recombinant chimeric IgE polypeptides with polyhistidine sequences can be recovered from a host cell homogenate by passing the homogenate over a nickel column and eluting the polyhistidine-containing polypeptides with imidazole. A particular recombinant chimeric IgE polypeptide with a leader sequence that directs that polypeptide's secretion can be recovered from the growth medium of a host cell expressing that polypeptide. For example, the growth medium from a culture of mammalian host cells expressing and secreting OSO polypeptides can be collected, and the OSO polypeptides can be recovered using chromatography. It is understood that a leader sequence that directs the secretion of a polypeptide typically is removed from that polypeptide in the host cell by proteolysis. Thus, the recovered secreted polypeptide, in many cases, is free of any translated leader sequence.

In one embodiment, the cell medium from a clonal CHO cell line expressing and secreting OSO polypeptides is collected and centrifuged to remove cell debris. After centrifuging, the supernatant is dialyzed and passed over an ion exchange column allowing the OSO polypeptides to bind. The bound OSO polypeptides are eluted using a sodium chloride/sodium acetate gradient, and the eluted fractions are screened for recombinant OSO polypeptides using an ELISA technique. The eluted fractions with high ELISA reactivity can be pooled and dialyzed again, and the dialyzed pooled fractions can be passed over a hydrophobic interaction column allowing the OSO polypeptides to bind. The bound OSO polypeptides are eluted using a sodium phosphate gradient, and the eluted fractions are again screened for recombinant OSO polypeptides using an ELISA technique. The eluted fractions with high ELISA reactivity can be further analyzed by silver stained SDS-PAGE to estimate the purity of the OSO polypeptides.

As described herein, alum as well as other aluminum-based compounds (e.g., Al₂O₃) can be combined with a polypeptide containing a human IgE polypeptide segment to form a composition that elicits an anti-human IgE response when administered to a human. Aluminum-based compounds can be obtained from various commercial suppliers. For example, ALHYDROGEL™, an aluminum hydroxy gel adjuvant, (Alhydrogel 1.3%, Alhydrogel 2.0%, or Alhydrogel “85”) obtained from Breuntag Stinnes Logistics can be used. REHYDRAGEL® adjuvants (Reheis Inc., Berkeley Heights, N.J.) also can be used. REHYDRAGEL® adjuvants are based on crystalline aluminum oxyhydroxide, and are hydrated gels containing crystalline particles with a large surface area (about 525 m²/g). Their Al₂O₃ content typically ranges from about 2 percent to about 10 percent. Rehydragel LG, for example, has an Al₂O₃ content of about 6 percent, and flows readily upon slight agitation. Rehydragel LG also has a protein binding capacity of 1.58 (i.e., 1.58 mg of bovine serum albumin bound per 1 mg of Al₂O₃), a sodium content of 0.02 percent, a chloride content of 0.28 percent, undetectable sulphate, an arsenic level less than 3 ppm, a heavy metal content less than 15 ppm, a pH of 6.5, and a viscosity of 1090 cp. Rehydragel LG can be combined with a polypeptide solution (e.g., a polypeptide in PBS) to yield Al(OH)₃.

Other adjuvants such as MN51 can be combined with a polypeptide containing a human IgE polypeptide segment to form a composition that elicits an anti-human IgE response when administered to a human. MN51 (MONTANIDE® Incomplete SEPPIC Adjuvant (ISA) 51) as well as MN720 are available from Seppic (Paris, France). MN51 contains mannide oleate (MONTANIDE® 80, also known as anhydro mannitol octadecenoate) in mineral oil solution (Drakeol 6 VR). MONTANIDE® 80 is a limpid liquid with a maximum acid value of 1, a saponification value of 164-172, a hydroxyl value of 89-100, an iodine value of 67-75, a maximum peroxide value of 2, a heavy metal value less than 20 ppm, a maximum water content of 0.35%, a maximum color value of 9, and a viscosity at 25° C. of about 300 mPas, MONTANIDE® associated with oil (e.g., mineral oil, vegetable oil, squalane, squalene, or esters) is known as MONTANIDE® ISA. Drakeol 6 VR is a pharmaceutical grade mineral oil. Drakeol 6 VR contains no unsaturated or aromatic hydrocarbons, and has an A.P.I. gravity of 36.2-36.8, a specific gravity at 25° C. of 0.834-0.838, a viscosity at 100° F. of 59-61 SSU or 10.0-10.6 centistokes, a refractive index at 25° C. of 1.458-1.463, a better than minimum acid test, is negative for fluorescence at 360 nm, is negative for visible suspended matter, has an ASTM pour test value of 0-15° F., has a minimum ASTM flash point of 295° F., and complies with all RN requirements for light mineral oil and ultraviolet absorption. MN51 contains about 8 to 12 percent anhydro mannitol octadecenoate and about 88 to 92 percent mineral oil. MN51 is a clear yellow liquid having a maximum acid value of 0.5, a saponification value of 16-20, a hydroxyl value of 9-13, a maximum peroxide value of 2, an iodine value of 5-9, a maximum water content of 0.5 percent, a refractive index at 25° C. between 1.455 and 1.465, a density at 20° C. of about 0.85, and a viscosity at 20° C. of about 50 mPaS. The conductivity of a 50:50 mixture of MN51 and saline is less than 10 μScm⁻¹.

Other adjuvants include immuno-stimulating complexes (ISCOMs) that can contain such components as cholesterol and saponins. ISCOM matrices can be prepared and conjugated to Cu²⁺ using methods such as those described herein. Adjuvants such as FCA, FIA, MN51, MN720, and Al(OH)₃ are commercially available from companies such as Seppic, Difco Laboratories (Detroit, Mich., and Superfos Biosector A/S (Vedbeak, Demark).

In some embodiments, a composition also can contain one or more additional immunostimulatory components. These include, without limitation, muramyldipeptide (e.g., N-acetylmuramyl-L-alanyl-D-isoglutamine; MDP), monophosphoryl-lipid A (MPL), and formyl-methionine containing tripeptides such as N-formyl-Met-Leu-Phe. Such compounds are commercially available from Sigma Chemical Co. (St. Louis, Mo.) and RIBI ImmunoChem Research, Inc. (Hamilton, Mont.), for example.

A “unit dose” of a composition refers to the amount of a composition administered to a human subject at one time. A unit dose of the compositions provided herein can contain any amount of polypeptide. For example, a unit dose of a composition can contain between about 10 μg and about 1 g (e.g., from about 10 μg to about 600 μg (e.g., from about 30 μg to about 600 μg, from about 100 μg to about 500 μg, from about 200 μg to about 400 μg, from about 250 μg to about 350 μg, from about 400 μg to about 600 μg, from about 450 μg to about 550 μg, or 10 μg, 15μg, 25 μg, 30 μg, 50 μg, 100 μg, 250 μg, 280 μg, 300 μg, 500 μg, 750 μg, 1 mg, 10 mg, 15 mg, 25 mg, 30 mg, 50 mg, 100 mg, 250 mg, 280 mg, 300 mg, 500 mg, 750 mg, or more) of a polypeptide. In some embodiments, the polypeptide can be dissolved or suspended in a physiological buffer such as, for example, water or phosphate buffered saline (PBS), pH 7.0. The solution of polypeptide then can be combined with an adjuvant and/or any other suitable component.

A unit dose of a composition can contain any amount of an adjuvant. For example, a unit dose can contain between about 10 μL and about 1 mL (e.g., 10 μL, 25 μL, 50 μL, 100 μL, 250 μL, 500 μL, 750 μL, 800 μL, 900 μL, or 1 mL) of one or more adjuvants. In addition, a unit dose of a composition can contain any amount of another immunostimulatory component. For example, a composition provided herein can contain between about 10 μg and about 1 g (e.g., 10 μg, 15 μg, 25 μg, 30 μg, 50 μg, 100 μg, 250 μg, 280 μg, 300 μg, 500 μg, 750 μg, 1 mg, 10 mg, 15 mg, 25 mg, 30 mg, 50 mg, 100 mg, 250 mg, 280 mg, 300 mg, 500 mg, 750 mg, or more) of an immunostimulatory component.

The compositions provided herein can contain any ratio of adjuvant to polypeptide. The adjuvant:antigen ratio can be 50:50 (vol:vol), for example. Alternatively, the adjuvant:antigen ratio can be, without limitation, 90:10, 80:20, 70:30, 64:36, 60:40, 55:45, 40:60, 30:70, 20:80, or 90:10.

This document also provides methods for preparing the compositions provided herein. Such methods can involve suspending an amount of a polypeptide (e.g., 100 μg of OSO) in a suitable amount of a physiological buffer (e.g., 50 μL of PBS pH 7.0), and then combining the suspended or dissolved antigen with a suitable amount of an adjuvant (e.g., 50 μL of MN51, or 100 μL of REHYDRAGEL® or ALHYDROGEL®). The combining step can be achieved by any method, including stirring, shaking, vortexing, or passing back and forth through a needle attached to a syringe, for example. It is noted that the composition can be prepared in batch, such that enough unit doses are obtained for multiple injections (e.g., injections into multiple animals or multiple injections into the same animal).

Also provided herein are methods for inducing an anti-human IgE response in a human. Such methods can involve administering to a human subject a composition provided herein, wherein the composition contains a polypeptide that includes an amino acid sequence from a human IgE polypeptide (e.g., an amino acid sequence from the CH3 domain of an IgE polypeptide found in humans). The polypeptide can contain at least one amino acid sequence from a species of non-placental mammal (e.g., an amino acid sequence from the CH2 or CH4 domain of an IgE polypeptide found in a species of non-placental mammal such as, for example, opossum).

In general, compositions containing a polypeptide provided herein can be used as an allergy vaccine to abrogate the allergic cascade by eliminating circulating IgE in a human recipient. The compositions can induce an antibody response against human IgE in the recipient. Although not limited to any particular mode of action, it is believed that administration of compositions containing a polypeptide with human IgE sequences in a context which allows the subject's tolerance to IgE to be broken leads to the production of anti-human IgE antibodies, which in turn decreases the level of circulating IgE antibodies.

The compositions provided herein can be administered by a number of methods. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g, by slow infusion or administration of slow release formulations).

Any dose can be administered to a human. Dosages can vary depending on the relative potency of individual compositions, and can generally be estimated based on data obtained from in vitro and in vivo animal models. Typically, dosage is from about 0.01 μg to about 100 g per kg of body weight, and may be given once or more daily, weekly, or even less often. Following successful administration, it may be desirable to have the subject undergo additional booster administrations to maintain a suitable level of the anti-human IgE response.

The anti-human IgE antibody response to a composition in a human subject can he assessed using any method. For example, the anti-human IgE titer can be measured. Alternatively, a “titer dilution₅₀ value” can be determined by using an ELISA and measuring the optical density (OD) of dilutions (e.g., serial dilutions) of the serum samples. The dilution factor that results in a 50 percent reduction from the maximal OD is considered to be the titer dilution₅₀ value. This value can be calculated by curve fitting using, for example, the SOFTmax® Pro 4.0 software program that is available from Molecular Devices, Inc. (Sunnyvale, Calif.). Using a four parameter non-linear regression for curve fitting, this program can be used to fit data points to a curve and determine the titer dilution₅₀ value.

This document also provides methods for measuring free IgE levels in the serum of a human subject treated with a polypeptide containing one or more human IgE segments (e.g., OSO). Such methods can involve providing a serum sample from a subject treated with, for example, OSO, and incubating the sample with an IgE receptor polypeptide (e.g., a recombinant IgE receptor polypeptide) such as the human IgE receptor alpha-chain (e.g., the polypeptide having GenBank® Accession No. NM_{—}002001) to form IgE/IgE receptor complexes. Any IgE receptor sequence (or portion thereof) can be used. For example, a human IgE receptor alpha-chain can be used to measure free IgE in humans. After incubating the IgE receptor polypeptide with the sample containing free IgE, the formed IgE/IgE receptor complexes can be measured. Any method can be used to measure IgE/IgE receptor complexes. For example, immunological assays such as ELISAs and ELISA-like procedures can be used to measure IgE/IgE receptor complexes.

In addition, this document provides kits for assessing the amount of free IgE present in a human subject treated with a composition described herein. Such kits can contain an IgE receptor sequence and an antibody capable of binding to an IgE/IgE receptor complex. The kits provided herein also can contain a composition described herein such as an OSO-containing composition. Such kits can be used to assess free IgE levels in a human subject and, if needed, to provide an additional booster of the human IgE polypeptide-containing composition. The kits provided herein can contain additional reagents such as IgE standards, negative controls, enzyme preparations, and enzyme substrates.

The invention will be further described in the following examples, which do not limit the scope of the invention described in the claims.

### EXAMPLE 1

### Materials and Methods

Dose escalation study to evaluate the safety and tolerability of anti-IgE immunotherapy: Thirty-two healthy human subjects were randomized into four dose groups of eight people each, with six subjects receiving a composition containing the active OSO drug product and two receiving placebo (saline). In addition to OSO, the composition contained ALHYDROGEL™ 1.3% (Brenntag Biosector, Frederikssund, Denmark), with one dose corresponding to 0.8 mg aluminum. Subjects received doses of 10 μg, 30 μg, 100 μg, or 300 μg OSO, independent of body weight. The four different dose levels were given in a semi-parallel sequential manner, so that the first injection of the consecutive dose level was given when the effect of the preceding dose level had been followed for 3 weeks and evaluated for safety and tolerability. Subjects were immunized at weeks 0, 3, and 5 with 10 μg, 30 μg, 100 μg, or 300 μg of OSO (the same dose was administered in all three immunizations). Serum samples were taken on days 1, 8, 15, 22, 29, 36, 43, 50, 57, 71, 85, and 99 (day 1 corresponds to week 0, day 8 to week 1, and so on), and were or will be taken at 18, 26 and 52 weeks. Final samples will be taken in May 2007.

Treatment with placebo served as a control for the influence of external factors on safety and pharmacodynamics. The study was double blinded to the study staff and the subjects. The blinding was broken for each group 14 weeks after the primary immunization, and the safety and tolerability of repeated injections of the anti-IgE immunotherapy were assessed. Pharmacodynamic profiles also were evaluated by assessing changes in levels of total IgE, unbound IgE, IgG anti-IgE, and IgG anti-OSO antibodies.

IgG anti-OSO ELISA: A semi-quantitative ELISA was used to measure human serum IgG anti-OSO levels (Landström, 2006, Resistentia Pharmaceuticals AB, Technical Report 06-31). Briefly, the ELISA was constructed as an indirect ELISA. Microtiter plates were coated with recombinant purified OSO produced in a CHO cell line. Serum samples were added and bound IgG anti-OSO was detected with goat anti-human IgG (Fc)-HRP (Jackson ImmunoResearch Laboratories, Inc., West Grove, Pa.).

The intra-assay variation was determined to 13% and inter-assay variation to 15%. All results are based on single determinations of serum samples. The mean of the blank readings was subtracted from standard and samples. The resulting optical densities of each dilution was fitted to a 4-parameter logistic equation and plotted in log/lin scale. The EC₅₀ values of standards and samples were used to define the IgG anti-OSO titer of each serum sample. Thus, EC₅₀ was defined as titer and reflects the number of dilutions needed to reach a 50% reduction of the optical density.

Time-points analyzed with anti-OSO ELISA were:
Dose group 1 (10 μg): weeks 0, 2, 4, 6, 8, 10, 12
Dose group 2 (30 μg): weeks 0, 2, 4, 6, 8, 10, 12
Dose group 3 (100 μg): weeks 0, 2, 4, 6, 8, 10, 12
Dose group 4 (300 μg): weeks 0, 2, 4, 6, 8 (n=4)*, 10 (n=4)* *Due to lag of 4 individuals of dose group 4

IgG anti-IgE ELISA: A semi-quantitative ELISA was used to measure human serum IgG anti-IgE levels (Fant, 2006, Resistentia Pharmaceuticals AB, Technical Report 06-40). Briefly, the ELISA was constructed as an indirect ELISA. Microtiter plates were coated with human IgE (HE1; Diatec.com AS, Oslo, Norway). Serum samples were added and bound IgG anti-IgE was detected with goat anti-human IgG (Fc)-HRP (Jackson ImmunoResearch Laboratories, Inc.).

Absorbance values (λ490 nm) at serum dilution 1:90 were used to determine the IgG anti-IgE response in duplicates.

Samples were analyzed in duplicates at the following time points:
Dose group 1 (10 μg): weeks 0, 3, 4, 5, 6, 7, 8, 10, 12
Dose group 2 (30 μg): weeks 0, 3, 4, 5, 6, 7, 8, 10, 12
Dose group 3 (100 μg): weeks 0, 3, 4, 5, 6, 7, 8, 10, 12
Dose group 4 (300 μg): weeks 0, 3, 4, 5, 6, 7, 8

IgG anti-IgE determined by a surface plasmon resonance assay (Biacore): A semi-quantitative response assay (Rydell, 2006, Resistentia Pharmaceuticals AB, Technical Report 06-38) was set up as an indirect assay using IgE immobilized to the to the sensor surface (CM5 Series S, Biacore AB, Uppsala, Sweden), and an injection of anti-IgG following injection of the sample, in order to enhance the response arising from anti-IgE antibodies of IgG-type. Briefly, the response of a sample was divided by the limit of detection (LOD) of that dose group. LOD was determined as the average of the relative response+3 standard deviations (SD) for the dose group at week 0 (LOD=Average+3*SD). Thus, a quota >1 was considered a positive response. An individual was considered having an IgG anti-IgE response if the response/LOD >1 at two consecutive time points.

Serum samples from the clinical trial were analyzed (Persson, 2006, Resistentia Pharmaceuticals AB, Techical Report 06-43) in duplicate on two different Biacore instruments, and thus were presented as the mean of quadruplicates±SD. The time points analyzed were the same as for the IgG anti-IgE ELISA.

In order to obtain information regarding the drift of the method, i.e., how the assay performed over time, a monoclonal IgG anti-IgE antibody (clone 95) was used instead of serum after each analyzed week. Any change in response for that monoclonal antibody between the first and last analysis can be interpreted as assay drift.

Total IgE: The total IgE was analyzed at Karolinska University Laboratory, Department of Clinical Immunology and Transfusion Medicine, Karolinska Institute, Stockholm, using a UniCAP 1000 instrument and the method UniCAP FEIA (Phadia, Uppsala, Sweden). All time points were analyzed.

Free IgE: Analyses of free IgE (unbound) were performed at the Johns Hopkins DACI Reference Laboratory, Baltimore, Md. The method was performed with slight modifications of that described in Hamilton et a. (J. Immunol. Methods, 2005, 303:81-91). This method was initially developed for measuring free IgE in allergic patients treated with a monoclonal anti-IgE antibody (Xolair®). Allergic patients typically have much higher levels of total IgE than healthy volunteers. At T=0 (week 0), the level of free IgE should equal the level of total IgE. However, deviations in the total IgE levels at T=0 were noted at free IgE levels below 90 U/mL. Therefore all T=0 measurements of free IgE lower than 90 U/mL were normalized to the total IgE of that patient for T=0. This T=0 correction factor was used for normalizing the week 6, week 8, and week 12 free IgE results. Free IgE levels below 90 U/mL should therefore be interpreted with caution. The limit of detection of the assay was 9 U/mL.

The following time points were analyzed:
Dose group 1 (10 μg): weeks 0, 6, 8, 12
Dose group 2 (30 μg): weeks 0, 6, 8, 12
Dose group 3 (100 μg): weeks 0, 6, 8, 12
Dose group 4 (300 μg): weeks 0, 6, 8

### EXAMPLE 2

### IgG anti-OSO ELISA

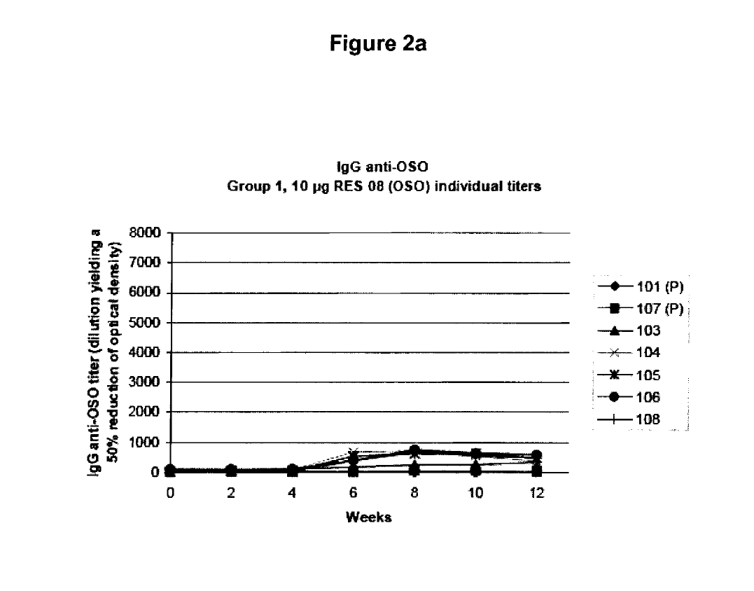

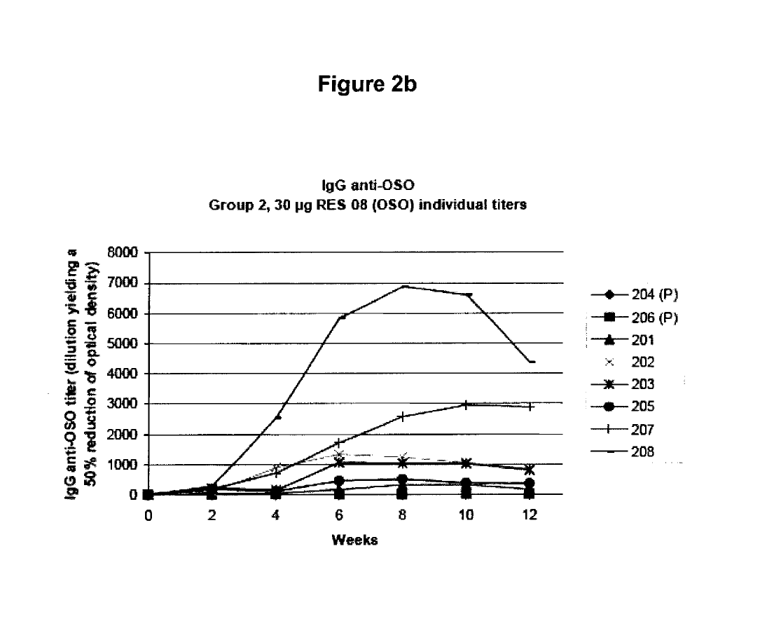

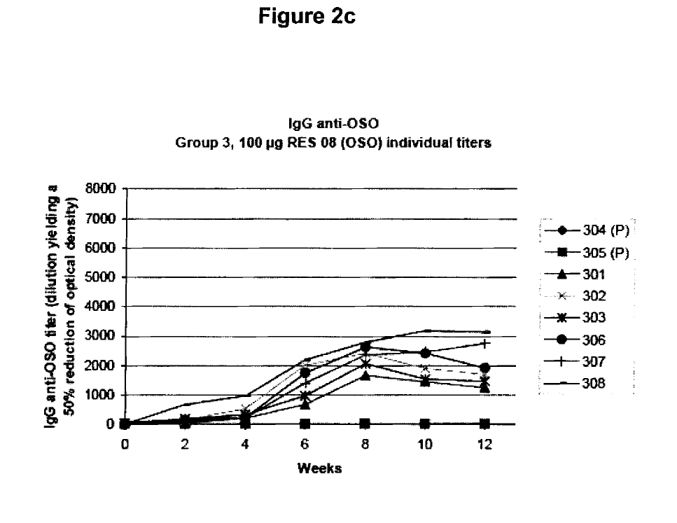

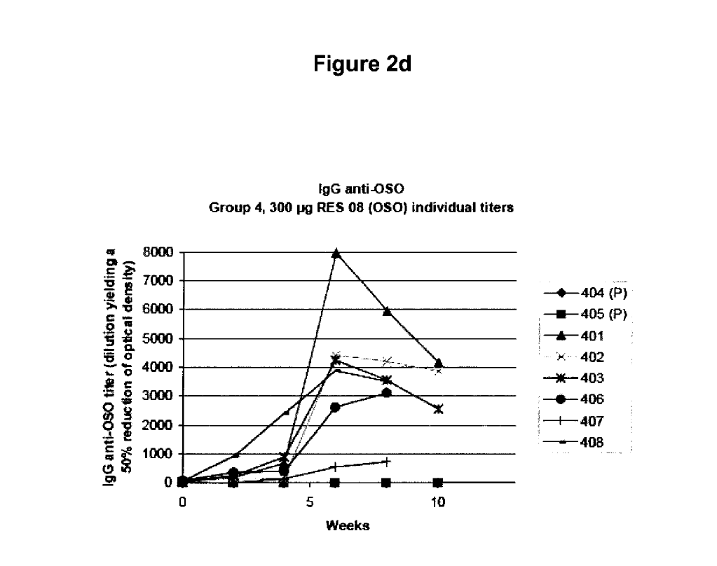

OSO was administered to human subjects as described in Example 1, and serum levels of IgG anti-OSO were measured by ELISA. FIG. 1 shows the group-wise IgG anti-OSO titers. A dose related increase of titers can be seen over time. The IgG anti-OSO response peaked at weeks 6-8, followed by a slight decline of the plateaus. FIGS. 2a-2d shows individual titer values of the different dose groups. The IgG anti-OSO response were found to be relatively homogenous within each group with the exception of subjects 208 (FIG. 2b) and subjects 401 and 407 (FIG. 2d). Neither serum samples at week 0 nor the placebo-treated individuals showed any response or cross reacting activity against OSO.

### EXAMPLE 3

### IgG anti-IgE ELISA

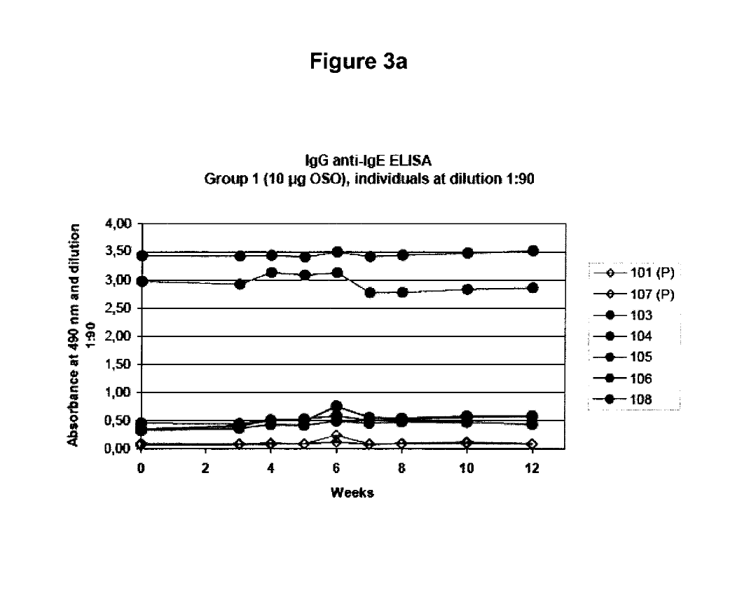

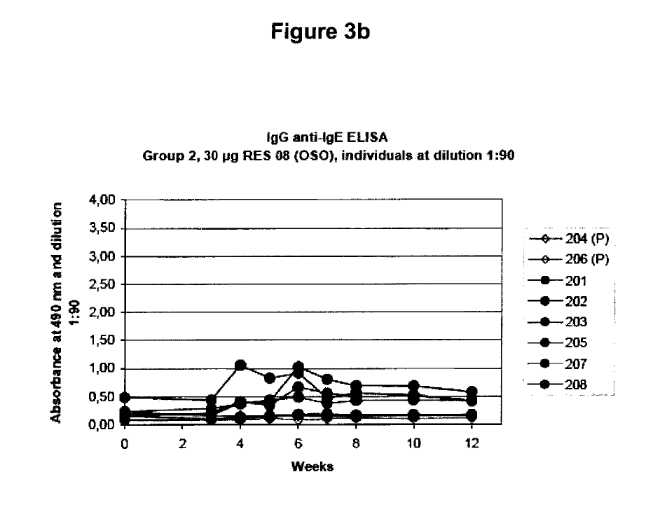

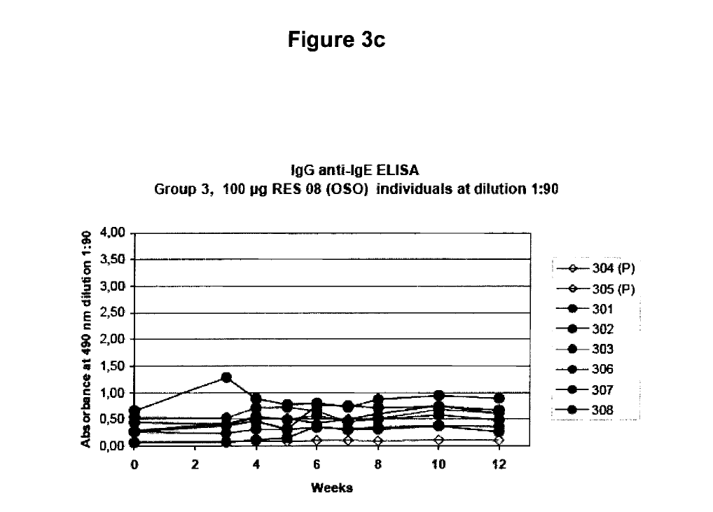

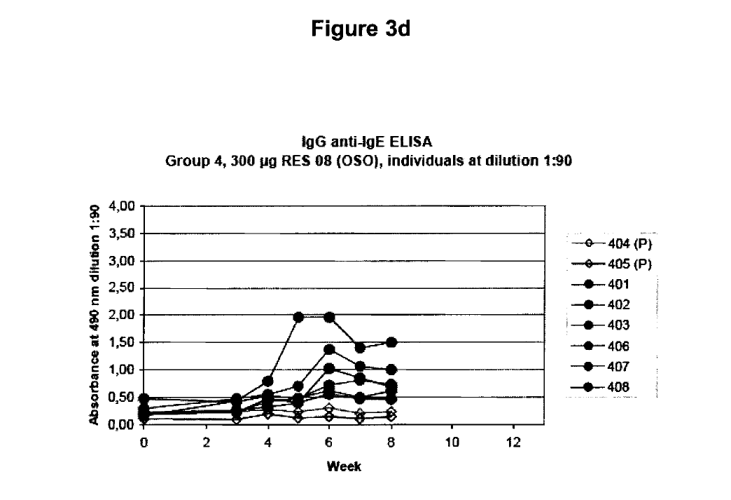

IgG anti-IgE was measured by ELISA as described in Example 1 and absorbance values (λ₄₉₀ nm) at dilution 1:90 of the sera were plotted. FIGS. 3a-3d depict the individual IgG anti-IgE results of the different dose groups. In general, a considerable variation of the response was observed among the individuals and dose groups. An absorbance above background (plate blank) was evident in all individuals pre-dose (week 0). In most individuals the absorbance was slightly above the background but in some subjects a clear signal was seen, suggesting the presence of IgG auto antibodies against IgE (Boluda et al, 1995, Clin. Exp. Immunol., 100:145-50; Lichtenstein et al., 1992, J. Immunol., 148:3929-3936; Ritter et al., 1991, J. Allergy Clin. Immunol., 88:793-801; and Twena et al, 1989, Clin. Immunol. Immunopathol., 53:40-51). In dose group 1 (FIG. 3a), the presence of auto antibodies against IgE was clearly observed in subjects 103 and 105, who had high absorbance values at pre-dose (week 0) and a sustained plateau over time. Subject 305, who was a placebo-treated individual (FIG. 3c), showed OD values suggesting the presence of auto antibodies against IgE. The presence of auto antibodies can make interpretation of IgG anti-IgE ELISA results complex. However, an unambiguous dose relationship was observed when comparing the different dose groups. The IgG anti-IgE response peaked between weeks 5-7 in the highest dose group (FIG. 3d).

### EXAMPLE 4

### IgG Anti-IgE Determined by a Surface Plasmon Resonance Assay (Biacore)

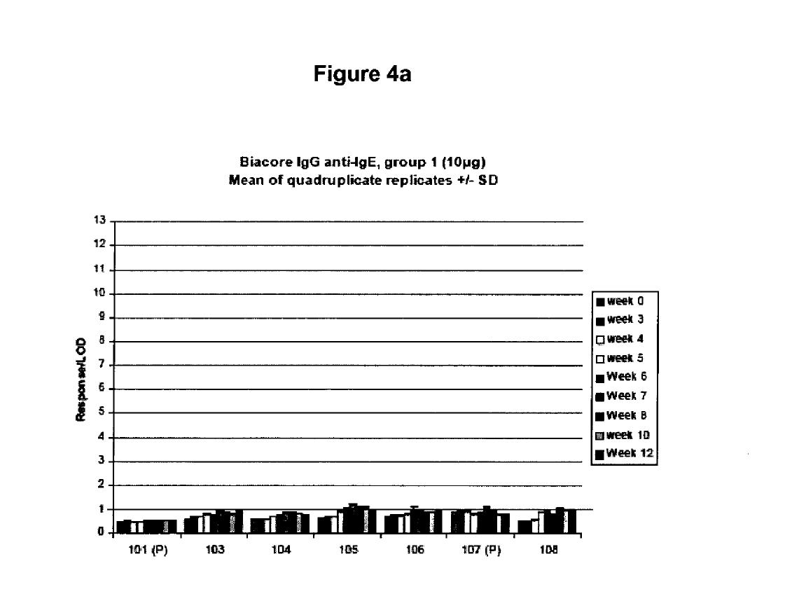

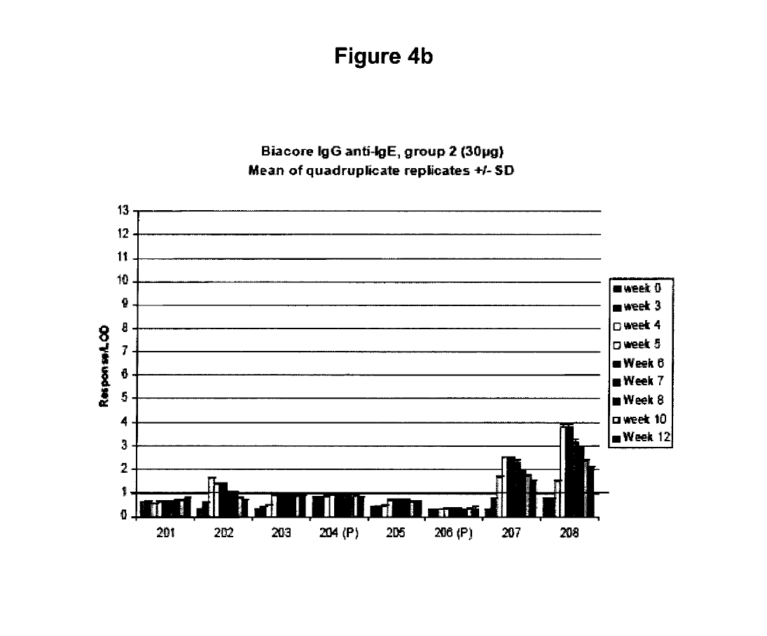

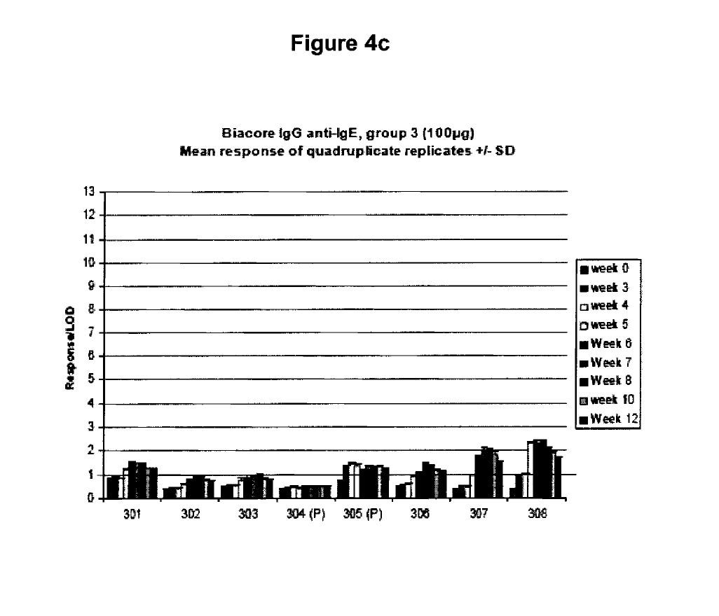

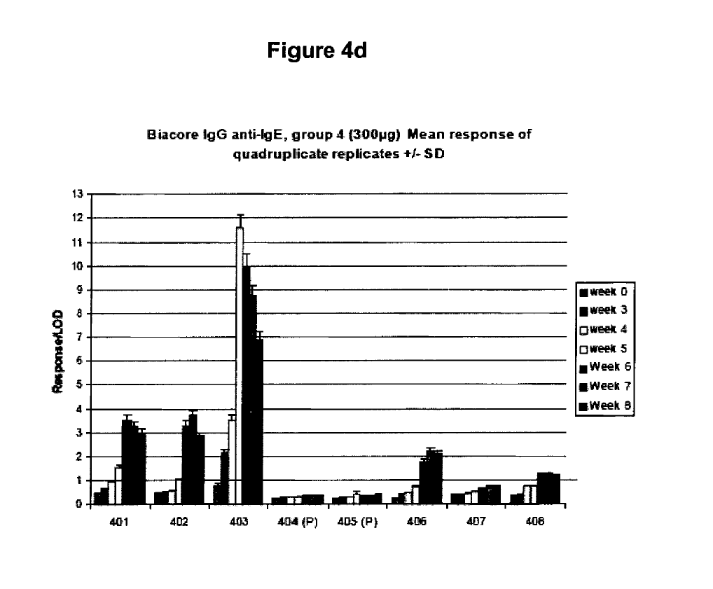

IgG anti-IgE surface plasmon resonance results were obtained with a biosensor (Biacore) as described in Example 1. FIGS. 4a-4d depict the IgG anti-IgE response in serum samples measured with the biosensor (Biacore). A response/LOD >1 was regarded as a positive IgG anti-IgE response, and individuals were considered to have an IgG anti-IgE response if the response/LOD >1 at two consecutive time points. Table 2 summarizes the number of individuals in each dose group with an IgG anti-IgE response according to this definition. An obvious dose related increase of the anti-IgE response was observed. Using the definition above, subject 305 (placebo-treated) also fell into the category of having an IgG anti-IgE response. The assay drift (decrease in activity from first to last cycle) ranged from 3.17% to 5.21%.

TABLE 2
Number of individuals with an IgG anti-IgE response/LOD > 1
on at least 2 consecutive time points
Biacore

| | |
|---|---|
| Group 1 (10 μg) | 1 |
| Group 2 (30 μg) | 3 |
| Group 3 (100 μg) | 4 (+1*) |
| Group 4 (300 μg) | 5 |

*Placebo

The Biacore method for measuring IgG anti-IgE is a relatively sensitive method, and also seems to be less influenced by the presence of auto antibodies than previously described methods. In the Biacore method, different IgE and detection antibodies were used compared with the IgG anti-IgE ELISA. This may account for the differences in detection of auto antibodies. Both IgEs and detection antibodies were used in the IgG anti-IgE ELISA, and both indicated the presence of auto antibodies. Thus, the Biacore may be less sensitive to the presence of auto antibodies.

Since LOD is based on relatively few samples (n=8 per dose group), a single sample that deviates to a large extent from the average can have a great impact on the LOD and may result in false positive IgG anti-IgE responses. Thus, an alternative method for data evaluation, in which outliers at week 0 are eliminated, may be useful. The presence of IgG-autoantibodies against IgE may explain the placebo treated individual having a response/LOD >1. The presence of auto antibodies has also been confirmed by ELISA. Further, the IgG anti-IgE ELISA results were in accordance with the results obtained using the Biacore method. Thus, two independent IgG anti-IgE immunoassays were in agreement, and indicated that the IgG anti-IgE immune response (both in frequency and magnitude) is dose dependent in doses ranging from 10 μg to 300 μg.

### EXAMPLE 5

### Total IgE and Free IgE

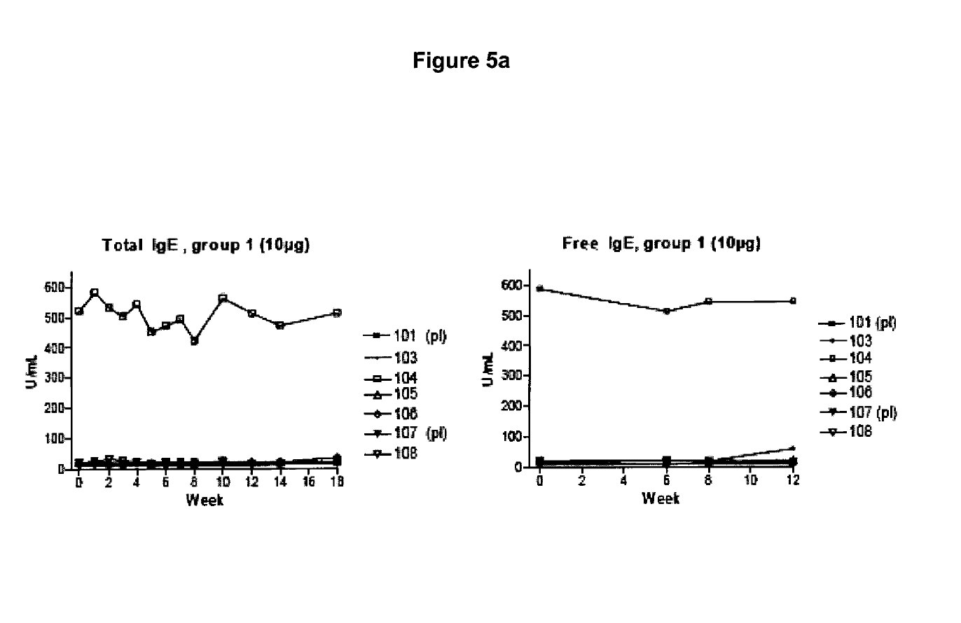

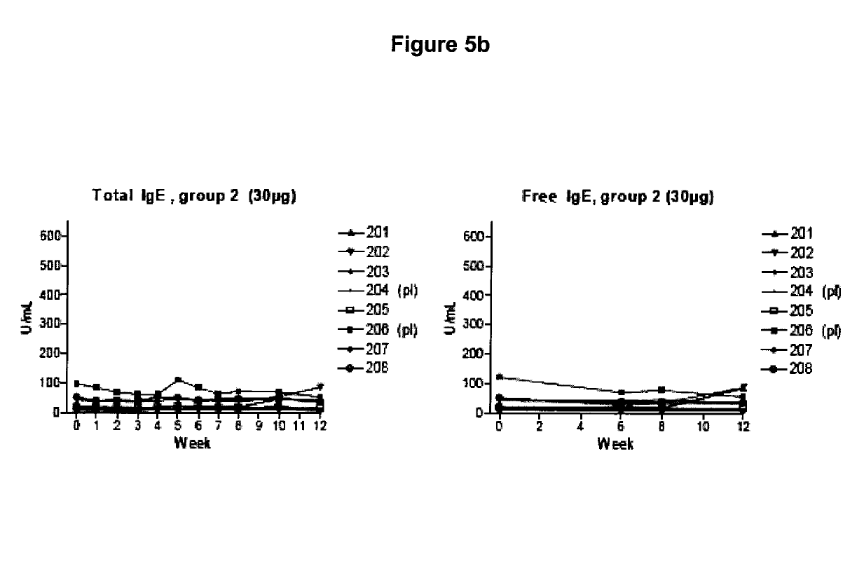

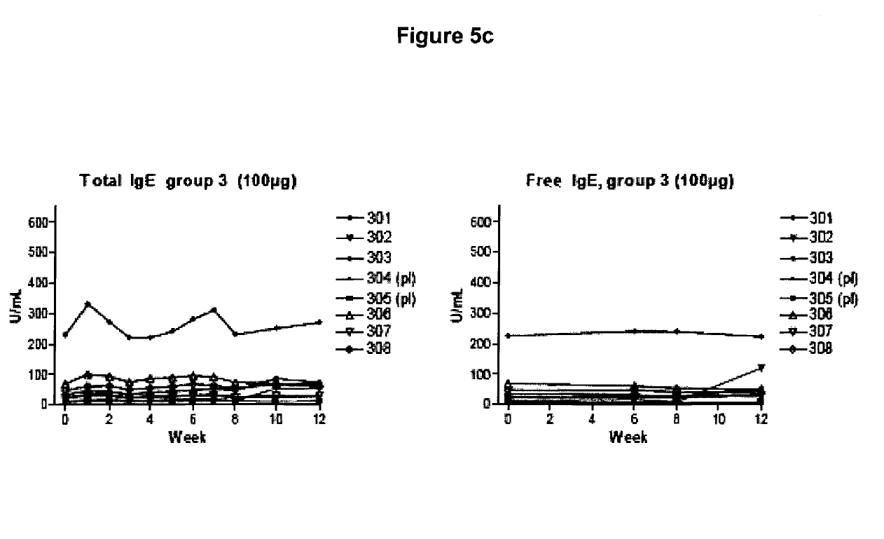

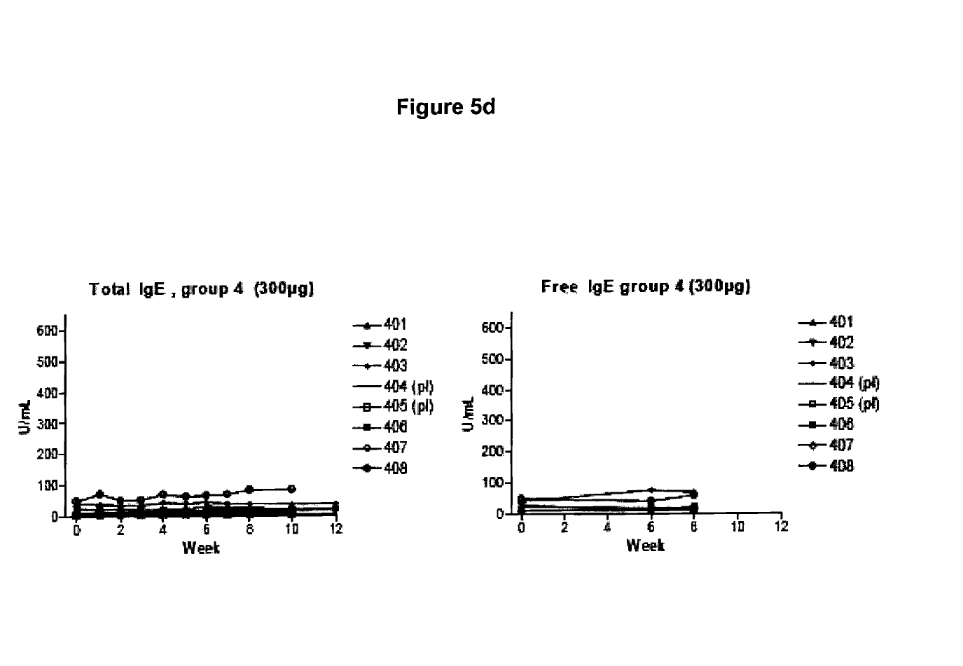

FIGS. 5a-5d summarize the total and free (unbound) IgE levels. Total IgE levels were within the normal range for all but three subjects (Kjellman et at., 1976, Clin. Allergy, 6:51-59), and no trend in the results was observed. Two subjects (104 and 301) had high levels of both total IgE and free IgE at all sampling times. The high levels of both total and free IgE were apparent despite that these subjects showed a negative phadiatope test at the screening visit, and have never experienced allergic symptoms. One subject (202) showed an increasing trend in both total and fee IgE from week 12. The reason for this increase was unclear, and the subject did not report any adverse events with a possible relationship to this IgE increase.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for inducing an anti-human IgE antibody response in a human, comprising administering to said human a polypeptide under conditions wherein said human produces an anti-human IgE antibody response, wherein said polypeptide comprises an amino acid sequence from a human IgE polypeptide and an amino acid sequence from an IgE polypeptide found in a non-placental mammal, and wherein said polypeptide is administered in an amount from about 30 μg to about 600 μg.

2. The method of claim 1, wherein said polypeptide is administered in an amount of about 30 μg.

3. The method of claim 1, wherein said polypeptide is administered in an amount of about 100 μg.

4. The method of claim 1, wherein said polypeptide is administered in an amount of about 300 μg.

5. The method of claim 1, wherein said polypeptide is administered in an amount of about 500 μg.

6. The method of claim 1, wherein said non-placental mammal is an opossum.

7. The method of claim 1, wherein said polypeptide comprises an amino acid sequence from a human IgE CH3 domain located between amino acid sequences from opossum IgE CH2 and CH4 domains.

8. The method of claim 1, wherein said polypeptide has the amino acid sequence set forth in SEQ ID NO:2.

9. The method of claim 1, wherein said administration induces a reversible anti-human IgE response in said human.

10. The method of claim 1, wherein said polypeptide is administered to said human under conditions wherein said human mounts an antibody response to human IgE that peaks and then decreases with time.

11. The method of claim 1, wherein said anti-human IgE response is a primary response that decreases with time.

12. The method of claim 11, wherein said primary response decreases to undetectable levels within 12 months.

13. The method of claim 1, wherein said administration induces an anti-human IgE response in said human after said human has experienced a primary anti-human IgE response.

14. The method of claim 13, wherein said polypeptide is be administered to said human under conditions wherein said human mounts an antibody response to human IgE in a manner consistent with a secondary antibody response.

15. The method of claim 1, wherein said administration induces a series of anti-human IgE responses in said human.

16. The method of claim 15, wherein said polypeptide is administered to said human at different times or under different conditions, wherein said human mounts a detectable anti-human IgE response that peaks within at least one year of each administration.

17. A composition comprising from about 30 μg to about 600 μg unit dose of a chimeric IgE polypeptide having the amino acid sequence set forth in SEQ ID NO:2.


SEQUENCE LISTING (2 sequences)

SEQ ID NO: 1 - DNA, 1014 bp, Artificial Sequence
  Chimeric Nucleic Acid
tcgagtactttatctctcccagaaagtggccctgtgacaatcatcccacctacagtgaagctcttccactcatcctgtgacccccgaggggatgctcattccaccatccagctgctctgccttgtctctggcttctccccagccaaggtccatgtgacctggctggtagatggacaggaggctgaaaatctctttccctatacaaccagacctaagagggaagggggacagactttttctctacaaagtgaagtcaacatcacacagggccagtggatgtcatcaaacacctacacctgccatgtcaagcacaatggcagcatctttgaagacagttctagaaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagcttgctagcccaggcaaacgcttagcccccgaggtatatatgctccctccatctccagaggaaacaggaaccactcgcactgtaacctgcctaattcggggtttctacccttctgaaatatctgtccaatggctgtttaataacgaagaggaccacactggacaccatactaccacccgtccccaaaaggaccacggaacggatccttccttcttcctctacagccgaatgcttgtcaacaagtctatttgggaaaaaggcaatctcgtcacctgccgtgtggtgcatgaagccctacctggctcccgcaccctggaaaaaagcctgcattactcagctggtaac

SEQ ID NO: 2 - protein, 338 aa, Artificial Sequence
  Chimeric Polypeptide
SSTLSLPESGPVTIIPPTVKLFHSSCDPRGDAHSTIQLLCLVSGFSPAKVHVTWLVDGQEAENLFPYTTRPKREGGQTFSLQSEVNITQGQWMSSNTYTCHVKHNGSIFEDSSRKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKLASPGKRLAPEVYMLPPSPEETGTTRTVTCLIRGFYPSEISVQWLFNNEEDHTGHHTTTRPQKDHGTDPSFFLYSRMLVNKSIWEKGNLVTCRVVHEALPGSRTLEKSLHYSAGN